(19) United States
(12) Patent Application Publication
(10) Pub. No.: US 2006/0069354 A1
(43) Pub. Date: Mar. 30, 2006

(54) Syringe activation device

(76) Inventors: David R. Buenger, Roswell, GA (US); Jeffrey T. Stout, Smyrna, GA (US); Carl E. Griffin, Marietta, GA (US)

Correspondence Address: GARDNER GROFF SANTOS & GREENWALD, P.C., 2018 POWERS FERRY ROAD, SUITE 800, ATLANTA, GA, 30339, US

(21) Appl. No.: 10/955,687

(22) Filed: Sep. 30, 2004

(51) Int. Cl. A61M 5/20; A61M 5/32

(52) U.S. Cl. 604/198; 604/157

(57) ABSTRACT

An apparatus for holding a syringe and injecting fluid from the syringe. The apparatus includes a barrel having a bore extending therethrough for receiving a body portion of the syringe, a drive spring, a drive mechanism movable under the influence of the drive spring to advance the syringe from a retracted position within the barrel to an advanced position wherein a needle portion of the syringe extends outwardly of the barrel, and to compress a plunger portion of the syringe relative to the body portion of the syringe to inject the fluid through the needle portion, and a retraction plunger movable within the drive sled between an advanced position and a retracted position, to permit the needle of the syringe to retract back into the housing after the injection is complete.

### TECHNICAL FIELD

The present invention relates generally to the field of medical injection aids for pharmaceutical delivery, and in particular to an injector pen for housing a syringe and delivering an injection to a user.

### BACKGROUND OF THE INVENTION

For those persons who require an injection of a pharmaceutical agent, such as insulin, allergy serum, or any number of vaccines, but fear being stuck with a needle, the injection process can be a stressful and painful experience. This ordeal can be exacerbated for those persons who require daily injections and/or who must frequently administer their own injections, such as diabetic persons. Some diabetic persons, such as children and elderly persons, experience difficulty in administering their own injections because of inexperience or lack of dexterity. Still others just simply fear the sight of the sharp needle.

Attempts have been made to provide injection aids, or injector pens as commonly referred to in the industry, that make the injection process easier. Typically, the user loads a prefilled, disposable syringe into the pen, places the pen against his or her skin at the injection site, and presses a button to administer the injection. However, many such injector pens have been found to be inadequate because they are quite cumbersome to use. For example, some injector pens are difficult and time consuming to load with a syringe. And because the sharp needle of a used syringe may project outwardly after the injection is administered from some previously known injector pens, a risk of inadvertent needle-sticks and transmission of dangerous blood-borne pathogens is present. Also, with some existing injector pens, there is a possibility that the syringe can inadvertently become dislodged from the pen during loading or removal, which could cause injury to the person using the pen.

Thus it can be seen that needs exist for an improved injection device and methods to facilitate the administering of a subcutaneous injection in a safe and easy manner and to prevent the inadvertent ejection of the syringe. Needs further exist for such mechanisms and methods that are readily adaptable for use with existing disposable syringes and that effectively hide the syringe from plain sight. It is to the provision of an improved injector pen meeting these and other needs that the present invention is primarily directed.

### SUMMARY OF THE INVENTION

In preferred forms, the present invention is an injector pen that accepts fixed and non-fixed needle syringes to facilitate injections, for example by persons who lack the manual and/or visual dexterity to operate standard syringes, or who have fear of syringe needles. The injector pen of the present invention is preferably re-usable with multiple syringes of standard commercial availability. In use, the user preferably does not see the needle during or after injection, helping to alleviate fear of needles. The device preferably positively engages the syringe to prevent inadvertent removal prior to injection, advances the needle of the syringe to penetrate the skin, fully dispenses the syringe's contents, and retracts the needle out of the skin and back into a shielded position.

In one aspect, the invention is an apparatus for holding a syringe and injecting fluid from the syringe. The apparatus preferably includes a barrel having a bore extending therethrough for receiving a body portion of the syringe; a drive spring; a drive sled movable under the influence of the drive spring to advance the syringe from a retracted position within the barrel to an advanced position wherein a needle portion of the syringe extends outwardly of the barrel, and to compress a plunger portion of the syringe relative to the body portion of the syringe to inject the fluid through the needle portion; and a retraction plunger movable within the drive sled between an advanced position and a retracted position.

In another aspect, the invention is an apparatus for activating a syringe, the apparatus preferably including a housing, a drive mechanism and a syringe retention mechanism. The syringe retention mechanism preferably includes a syringe retention piston and at least one syringe retraction spring.

In another aspect, the invention is an apparatus for activating a syringe, the apparatus preferably including a housing, a drive mechanism and a syringe retention mechanism. The drive mechanism preferably includes at least one drive spring for moving a drive sled between an advanced position and a retracted position within the housing, and a retraction plunger movable between an advanced position and a retracted position within the drive sled.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 shows a perspective view of an apparatus for housing and actuating a syringe and injecting a pharmaceutical fluid therefrom, according to one example embodiment of the present invention, shown in partial cross-section to more clearly show internal components.

FIG. 2 shows the apparatus of FIG. 1, with a drive mechanism thereof in a charged position.

FIG. 3 shows the apparatus of FIG. 1, with a retraction plunger portion thereof engaging the syringe.

FIG. 4 shows the apparatus of FIG. 1, with its retraction plunger advancing the needle of the syringe out of the apparatus.

FIG. 5 shows the apparatus of FIG. 1, with the retraction plunger compressing the syringe plunger to inject a pharmaceutical fluid.

FIG. 6 shows the syringe retraction mechanism of the apparatus of FIG. 1 in greater detail, with its retraction plunger retracted to allow retraction of the syringe once the injection has been completed.

FIG. 7 is a top plan view of an apparatus for housing and actuating a syringe and injecting a pharmaceutical fluid therefrom, according to another example embodiment of the present invention.

FIG. 8 is a perspective view of the apparatus of FIG. 7, with a portion of its housing removed to show internal components.

FIG. 9 is a perspective view of a receiver portion of the apparatus of FIG. 7 for engaging a syringe therein.

FIG. 10 is a cross sectional view of the apparatus of FIG. 7, with a piston portion thereof partially extended into engagement with a syringe and advancing the needle of the syringe out of the apparatus.

FIG. 11 is a cross sectional view of the apparatus of FIG. 7, with the piston portion fully extended and compressing the plunger of the syringe to administer a pharmaceutical fluid.

FIG. 12 is a cross sectional view of the apparatus of FIG. 7, with a plunger portion thereof retracted to permit withdrawal of the syringe needle back into the barrel.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

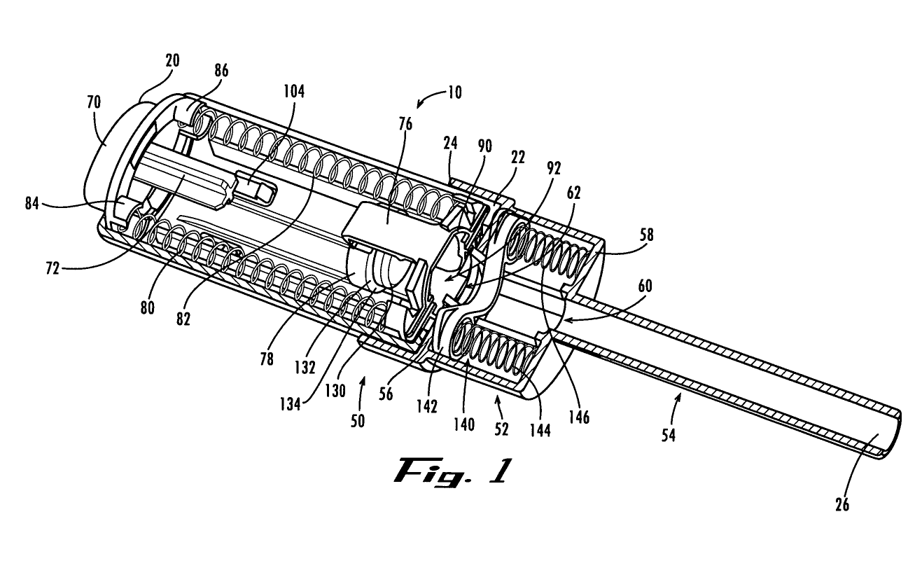

With reference now to FIGS. 1-6, an injection apparatus 10 suitable for housing a disposable syringe 12 and delivering a pharmaceutical fluid therein to a patient is shown in accordance with an example embodiment of the present invention. Preferably, the device 10 is reusable with multiple syringes, but alternately, the device 10 can be a single-use device that is discarded after use with one syringe. The device 10 has a housing 14 including a handle portion 16 having a drive mechanism housed therein, and a lower portion 18 having a syringe receiver portion with a hollow tubular barrel extending therefrom. As depicted in FIG. 1, the overall shape of the housing 14 preferably resembles that of a screwdriver or a pen. The housing 14 is preferably opaque, and the length of the housing is preferably long enough to fully enclose a standard, disposable syringe loaded with a pharmaceutical fluid, without having any portion of the syringe externally visible when the apparatus 10 is used.

The handle portion 16 of the housing preferably has a proximal end 20 and a distal end 22, and the lower portion 18 of the housing has a proximal end 24 and a distal end 26 defining an opening. The distal end 22 of the handle 16 preferably is permanently or releasably connected to the proximal end 24 of the lower portion 18, via a threaded, press-fit or adhesive connection. At least the lower portion 18 of the housing is openable, as for example by a hinged clamshell housing structure, to permit a syringe to be loaded into the apparatus 10 for use and removed for disposal after use.

A typical syringe 12 suitable for use with the apparatus 10 of the present invention has a generally cylindrical body 40 for containing a quantity of pharmaceutical fluid therein, a plunger 42 that slides within the cylindrical body, and a fixed or removable needle 44 for penetrating the skin and delivering a subcutaneous injection to a patient. A pair of finger stops or flanges 46, 48, typically project from opposing sides of the body 40, near the open end of the body into which the plunger 42 is received, to assist the user in drawing the pharmaceutical fluid into the syringe 12 and injecting the fluid typically by allowing the user to press his or her middle and index fingers against the stops 46, 48, while using his or her thumb to compress the plunger 42 into the cylindrical body 44. The apparatus 10 of the present invention is preferably adaptable for use in connection with any of a variety of standard commercially-available syringe types.

Preferably the first portion 16 of the housing 14 has a somewhat flattened cylindrical shape such that a cross-section taken perpendicularly to the width of the housing would yield a generally oval shape. The lower portion 18 is preferably an integral component having three stepped or tiered sections, a connector section 50 for connection to the handle 16, a receiver section 52 for engaging a syringe, and a hollow tubular barrel 54. An upper receiver wall 56 preferably extends across the interior of the lower portion 18 between the connector section 50 and the receiver section 52, and a lower receiver wall 58 extends between the receiver section and the barrel 54. An opening 62 is preferably provided through the upper receiver wall 56 for allowing passage of the syringe plunger into the handle 16 for engagement with the drive mechanism as described below. The lower receiver wall 58 preferably also defines a central opening 60, communicating with the bore of the barrel 54, through which the body of the syringe passes. The internal bore of the barrel 54 preferably has an inner diameter that is slightly larger than the outer diameter of the syringe to be loaded therein, so as to allow the syringe to slide smoothly therein during advancement and retraction when the device is actuated.

Within the receiver section 52 of the second portion 18 of the housing 14 is a syringe retention mechanism 140, as more clearly depicted in FIG. 6. The syringe retention mechanism 140 comprises a syringe retention piston 142 and at least one, and preferably, a pair of springs 146, 148 or other biasing elements. The retention piston 142 preferably has a generally U-shaped profile with a recess 144 therein to receive the cylindrical body 40 of the syringe 12. One end of each spring 146 and 148 abuts the inner surface of the lower receiver wall 58 and the other end abuts the surface of the syringe retention piston 142. The biasing elements 146, 148 act in compression to bias the syringe retention piston 142 in a rearward direction towards the upper receiver wall 56. Preferably, the finger stops 46 and 48 rest on the top surface of the syringe retention piston such that when the springs 146, 148 bias the syringe retention piston 142 towards the upper receiver wall 56, the finger stops 46 and 48 are securely engaged between the upper receiver wall and the piston. Additionally, the syringe retention piston 142 continues to retain contact with the syringe 12 as it is driven towards the open end 26 of the device 10 in use.

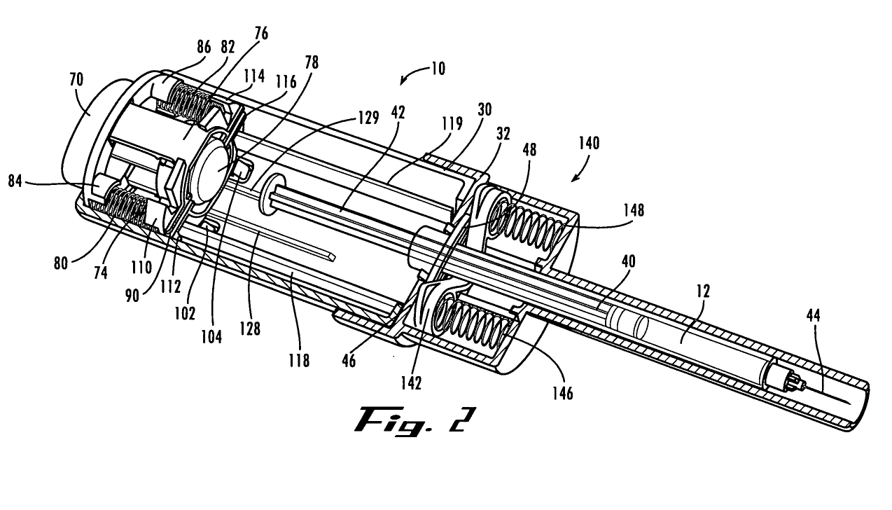

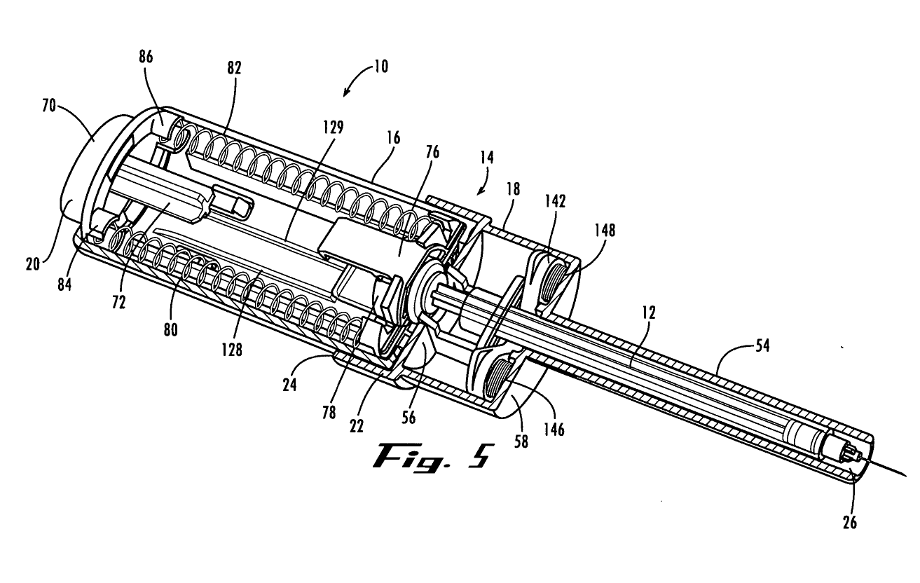

The apparatus 10 preferably further comprises a drive mechanism 74 housed within the handle 16 of the housing. The drive mechanism preferably comprises a drive carriage or sled 76, a retraction plunger 78 engaged within the drive sled, and one or more drive springs 80, 82 or other advancing mechanism for driving the drive sled along its stroke of travel upon activation. An actuation button or trigger 70 is preferably located at the proximal end 20 of the handle 16 for actuating the device 10 to release the drive sled 76 from an energized position with drive springs 80, 82 loaded in compression between the activation button 70 and the drive sled, as shown in FIG. 2, to an extended position as shown in FIG. 5.

The actuation button 70 preferably has a plunger setting shaft 72 extending toward the distal end of the apparatus. The shaft 72 preferably has a cross-shaped cross-section, although other cross-sectional profiles can alternatively be employed. The plunger setting shaft 72 contacts the retraction plunger 78 to press it forward and set it in an advanced position in the drive sled when the drive sled is pressed rearwardly to arm the device.

Preferably, the drive sled 76 has a generally square-shaped front panel 90 having an opening 92 therethrough in about the center of the panel 90, and four corners. 94, 96, 98, and 100. One or more, and preferably four retention tabs (two of which are shown in the figures) 102, 104 preferably are provided on the interior of the first portion of the housing, to engage the panel 90 near its corners 94, 96, 98, and 100, and hold the drive sled 76 in a retracted position. As shown in the figures, each retention tab is located within a cutout section of the first portion 16 of the housing 14. The retention tabs 102, 104 are preferably J-shaped, having barbed free ends for holding the sled 76 in a retracted position when the device is armed or cocked to energize the drive mechanism, and are flexible such that when the actuation button 70 is pressed inwardly, the force of the shaft 72 pushing on the drive sled 76 causes the retention tabs to flex away from the drive sled and into the wall of the housing 14, which, in turn, releases the drive sled to traverse its stroke of travel.

The drive sled 76 also preferably has a pair of flexible arms 120, 122 extending rearwardly towards the proximal end of the device 10 that retain and engage the retraction plunger 78 as it is advanced and retracted within the drive sled. Preferably, the retraction plunger 78 is a generally cylindrical element that is movable within the drive sled 76 between an advanced position and a retracted position. In the advanced position (FIGS. 2-5), a narrow front portion 130 of the plunger 78 protrudes through the opening 92 in the front panel 90 of the drive sled, and a wide rear portion 132 abuts the rear face of the panel 90 to hold the plunger in place within the drive sled.

Preferably, the arms 120, 122 have inwardly curved or barbed free ends 123, 124, that retain the retraction plunger 78 therebetween when the retraction plunger is in its retracted position, as depicted in FIGS. 1 and 6. Also preferably, the arms 120, 122 each have an intermediate barb 125, 126 that retains the retraction plunger 78 in its advanced position, with its front portion extending through the opening 92 in the front panel of the drive sled until the plunger 42 of the syringe 12 is fully compressed. Also, the arms 120, 122 are flexible in that they flex outwardly against the housing 14 to retain and release the retraction plunger during operation.

The handle 16 preferably also has two pairs (only one of which is shown) of plunger retention tracks 128, 129, which exert an inward force on the arms 120 and 122, to keep the arms flexed inwardly (i.e., away from the housing) to retain plunger 78 in its advanced position in the drive sled 76 as the drive sled advances to compress the plunger of the syringe. The retention tracks 128, 129 preferably terminate a distance from the distal end 22 of the handle 16 that is approximately equal to the length of the drive sled 76. Thus, when the sled 76 reaches its advanced position and injection from the syringe is completed, the arms 120, 122 of the drive sled are allowed to flex outwardly against the housing and thereby release the retraction plunger 78 from engagement with the intermediate barbs 125, 126. Once released from the intermediate barbs 125, 126, the plunger 78 retracts between the arms 120, 122 until it engages the inwardly directed ends 123, 124 of the arms. Thus once the injection process is complete and when the sled 76 reaches its fully advanced position, the retraction plunger 78 retracts, which in turn allows the syringe 12, under the influence of springs 146 and 148, to retract into the housing.

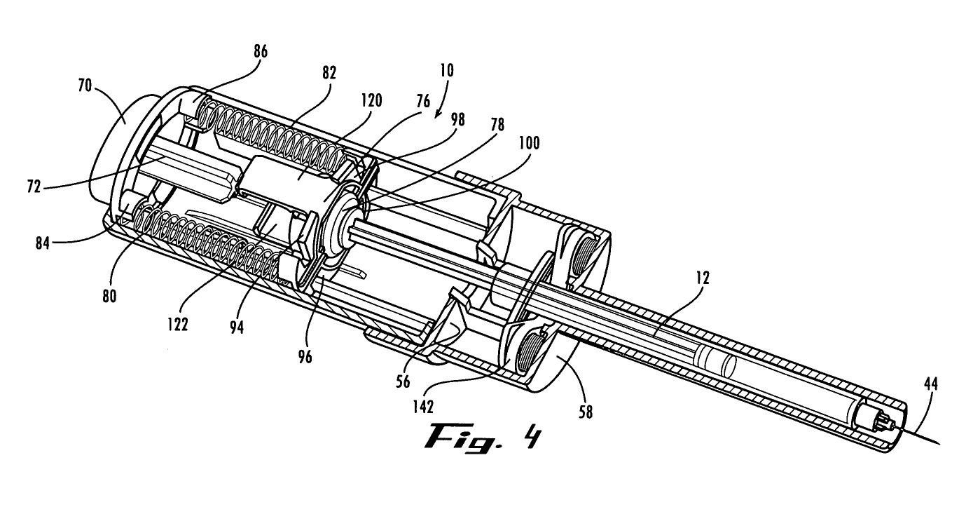

Because the force required to overcome the rearward bias of springs 146, 148 and advance the syringe 12 through the barrel 54 is preferably less than the force required to compress the plunger of the syringe and dispense the syringe's contents, the coupled drive sled 76 and retraction plunger 78 preferably extend under the influence of drive springs 80 and 82 to first advance the needle 64 of the syringe outwardly through the opening in the end of the barrel and into the subject's skin (FIG. 4), and then continue extending to compress the plunger of the syringe and inject the syringe's contents. Upon reaching its fully extended position wherein the contents of the syringe 12 have been fully dispensed (FIG. 5), the retraction plunger 78 is preferably released from engagement with the intermediate barbs 125, 126 as the arms 120, 122 of the sled 76 move beyond the ends of the plunger retention tracks 128, 129, and are allowed to flex outwardly. Because the plunger of the syringe is fully compressed and the syringe body and retention piston 142 are advanced into abutment with the lower receiver wall 58, the back of the syringe plunger presses the retraction plunger rearwardly to release it from its advanced position to its retracted position (FIG. 6). As the retraction plunger retracts into contact with the barbed ends 123, 124 of the arms 120, 122 of the drive sled 76, the syringe retracts and the syringe needle 64 is withdrawn under the influence of the springs 146 and 148, out of contact with the subject's skin and into a retracted position shielded within the barrel 18 to prevent inadvertent contact with the contaminated sharp needle.

Also preferably, along two opposing ends of the face 90 of the drive sled are pairs of guides 110, 112 and 114, 116 that engage and slide along four guide tracks (only two of which are shown) 118, 119 on the interior of the handle 16 of the housing 14. As shown in the figures, guide 112 engages track 118 and guide 116 engages track 119. The other two guides 110 and 114 engage the tracks not shown in the other half of the handle 16. The interaction between the guides and the guide tracks assists the drive sled 76 in moving smoothly in a linear advancement direction to engage the syringe 12 and to drive and compress the plunger of the syringe in a linear direction.

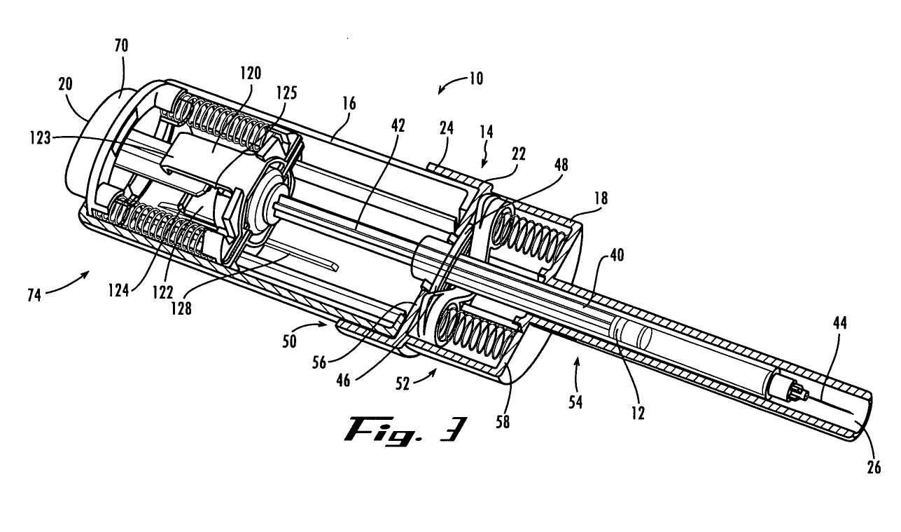

In operation, the user preferably opens a clamshell receiver portion of the housing and loads a syringe 12 prefilled with a pharmaceutical fluid into the syringe engagement mechanism 140 of the device 10. The device is energized to compress the drive springs 80, 82 and engage the drive sled 76 with retention tabs 102, 104 in its retracted position, as shown in FIG. 2. Once the syringe 12 has been loaded, the user closes the receiver portion of the housing. The user places the open end 26 of the device 10 against his or her skin at the desired injection site. The user actuates the device 10 by pressing the actuation button 70 inwardly. The force acting on the shaft 72 of the inwardly pressed actuation button 70 causes the drive mechanism 74 to become disengaged from the retention tabs and drives the drive mechanism 74 towards the distal end 26 of the device 10. The drive sled advances into contact with the syringe, as depicted in FIG. 3. The drive springs 80, 82 acting on the drive sled 76 overcome the rearward bias of syringe retraction springs 146, 148, advancing the syringe 12 towards the distal end 26 of the device and out of the opening in the end of the barrel to cause the needle 64 to penetrate the user's skin. Continued advancement of the drive sled 76 drives the plunger 62 of the syringe 12 into the body 60 of syringe 12, which in turn, injects the pharmaceutical fluid therein to the user. As the drive sled reaches its fully advanced position, it disengages from the plunger retention tracks 128 and 129, and the arms 120, 122 of the sled flex outwardly against the housing to release the retraction plunger 78 from engagement with the intermediate barbs 125, 126. The retraction plunger 78 retracts within the drive sled 76 and is retained in its retracted position by the barbed ends 123, 124 of the arms 120, 122 of the drive sled. The advancing bias of the drive sled 76 on the syringe 12 is thereby relieved, and the syringe retraction springs 146, 148 retract the syringe, drawing needle 64 back into the housing 14 of the device 10. The injection is now complete, and the user can open the housing to remove and dispose of the spent syringe 12. The device can be reloaded with a fresh syringe, and the drive sled 76 and plunger 78 can be pushed back into engagement contact with the retention tabs 102 and 104, for example by inserting end 26 of the barrel 54 into the handle 16 to compress the drive springs 80, 82 and re-cock the device.

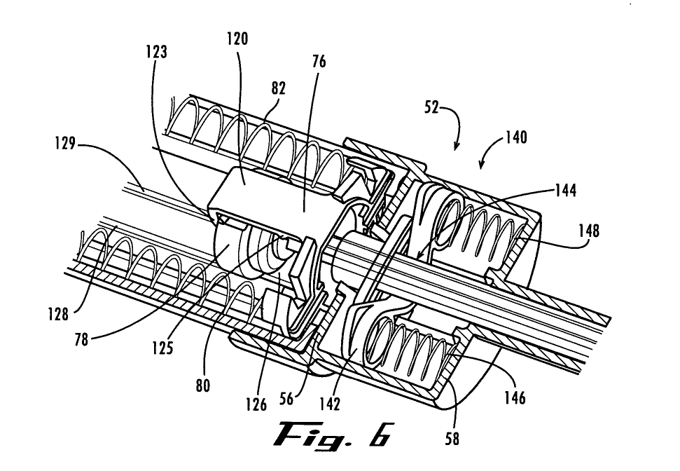

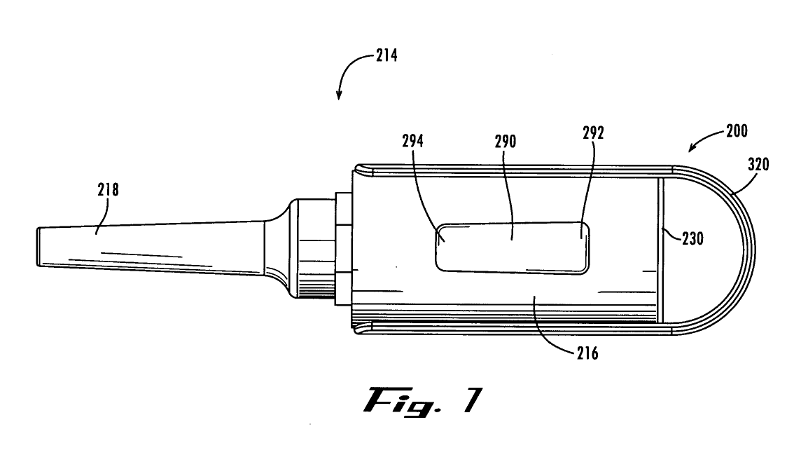

With reference now to FIGS. 7-12, another example embodiment of an injection pen 200 suitable for housing a syringe 12 and delivering a pharmaceutical fluid to a user is shown in accordance with the present invention. Preferably, the device is reusable, but alternately, the device can be a single-use device that is discarded after use. The device 200 has a housing 214 including a handle portion 216 and a barrel portion 218 releasably secured to the handle portion. Preferably, the housing 214 is constructed of an opaque material, such as an opaque plastic, that prevents the syringe from being visible to the subject during the injection process. The overall shape of the housing 214 generally resembles that of a pen or a screwdriver, and the length of the housing 214 is preferably long enough to accommodate a standard, disposable or re-usable syringe, without having any portion of the syringe extending outwardly of the housing. Thus, once loaded into the device, the syringe 12 is wholly contained within the housing 214 and cannot be seen through the housing 214.

The handle portion 216 has a first or proximal end 220 and a second or distal end 222, and the barrel portion 218 has a first or proximal end 224 and a second or distal end 226 defining a needle opening. The distal end 222 of the handle portion 216 preferably adjoins and connects with the proximal end 224 of the barrel portion 218. Preferably, the handle and barrel portions of the housing 14 is releasably coupled in a manner similar to that of the first embodiment via a threaded connection; or alternately, other fastening methods, including but not limited to, a snap connection, press-fit, or adhesive connection can be employed as well. Optionally, at least a portion of the housing is openable, as for example by a hinged clamshell housing structure or by detachment of a receiver portion of the barrel from the handle, to permit a syringe to be loaded into the apparatus 200 for use and removed for disposal after use.

Preferably, the handle portion 216 of the housing 214 is generally cylindrical or prismatic in shape, and has a cap 230 permanently affixed at its proximal end 220. Secured to the inner face of the cap 230 is a cylindrical hub 232 for engaging and reseating a piston portion 282 of the drive mechanism 280. The barrel portion 218 is preferably also generally cylindrical, comprising a hollow tubular member having an internal bore extending lengthwise therethrough. In the depicted embodiment, the barrel portion 218 is a continuous piece having three tiered sections, a first section 242, a second section 244, and a third section 246. The outer diameter of the first section 242 is preferably approximately equal to the inner diameter of the handle portion 216 of the housing 214 so that the two portions can readily couple together. The diameter of the first section 242 is preferably larger than the diameter of the second section 244, which is preferably larger than the diameter of the third section 246, thus defining a stepped external housing configuration. In alternate embodiments, a smoothly tapered configuration is provided. The inner bore diameter of the third section 246 is preferably slightly larger than the outer diameter of the syringe 12 to allow the syringe 12 to smoothly advance therein toward the injection site without impeding the syringe's movement.

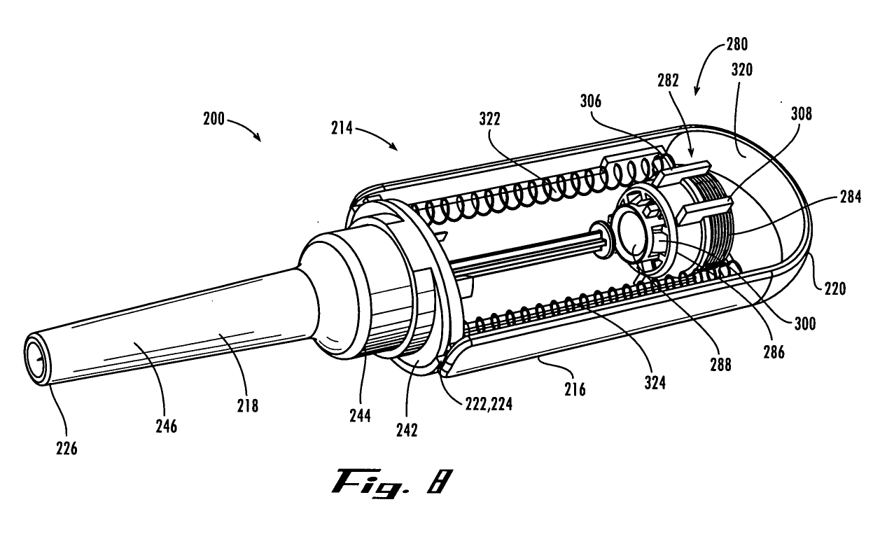

Referring now to FIG. 9, the injection apparatus 200 preferably includes a receiver 260 within the second section of the barrel portion for receiving and retaining the syringe 12. Preferably, the receiver 260 comprises a collar 272, and a syringe abutment flange 274 having a cylindrical passage (not shown) therethrough for receiving the cylindrical body 60 of the syringe 12. The syringe abutment flange 274 preferably defines an elongate recessed area 276 for receiving the finger flanges 66, 68 of the syringe such that the finger flanges rest within the recessed area. A return spring 278 or other biasing element preferably acts in compression between an interior shelf 250 between the second and third sections 244, 246 of the barrel portion and the syringe abutment flange 274, and biases the syringe abutment flange 274 towards the proximal end of the barrel into abutment against the collar 272.

The injection apparatus 200 preferably also includes a drive mechanism 280 and an actuation member or trigger 290 for releasing the drive mechanism and actuating the device to deliver the pharmaceutical fluid from the syringe 12 to the user. The trigger 290 preferably comprises a lever arm that extends from a first end 292 near the proximal end 220 of the handle portion 216 to a second end 294 near the distal end 222 of the handle. Near the first end 292 of the lever 290 is a fulcrum 296. A biasing element 298, for example a spring, biases the second end 294 outwardly from the device 200, which in turn biases the first end 292 of the lever 290 inwardly. The first end 292 of the lever 290 is biased inwardly through an opening in the housing 214 to engage the drive mechanism 280 and retain the drive piston 282 in its retracted position when the device 200 is in its charged or loaded position. When the trigger 290 is actuated, the drive mechanism 280 is released to advance the syringe needle through the subject's skin, and to compress the plunger to inject the contents of the syringe.

Preferably, the drive mechanism 280 includes a piston portion 282 and a drive spring 284 provided in compression between the cap 230 of the handle 216 and the piston portion to drive the piston portion from the retracted position shown in FIG. 8 to advance the syringe and inject its contents. The piston portion 282 itself includes an outermost drive sled 286 and a retraction plunger 288 engaged within the sled. The plunger 288 has at least one cantilevered arm 300 for engaging a seat 302 of the sled 286, which holds the retraction plunger within the sled as the sled is driven forwardly. In the depicted embodiment, three cantilevered arms are shown. Preferably, the drive sled 286 has a shelf 304 at its proximal end for engaging the retraction plunger in its retracted position within the sled and retaining the retraction plunger 288 therein. Also, preferably, the retraction plunger 288 has a front contact surface for advancing into contact with the back of the plunger 62 of the syringe 12 so as to compress the plunger into the cylindrical body 60 of the syringe when the device is actuated. On the outer surface of the drive sled is at least one, and preferably a plurality of fins 306, 308 (two of which are shown in the depicted embodiment) that slide within cooperating tracks on the inner surface of the handle portion 216, to guide the drive mechanism 280 along a linear advancement and retraction path.

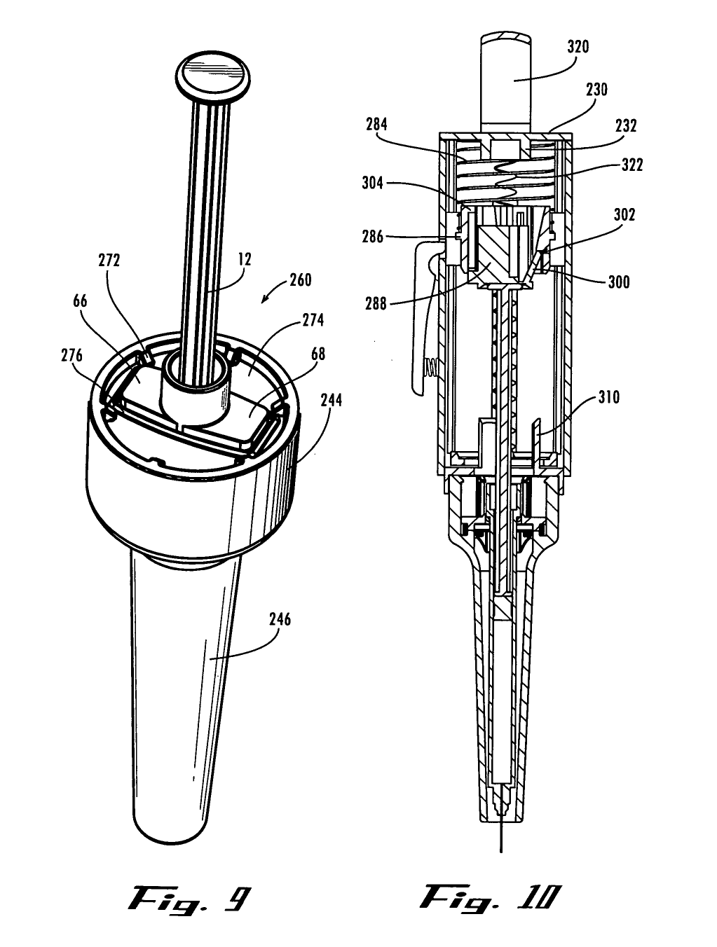

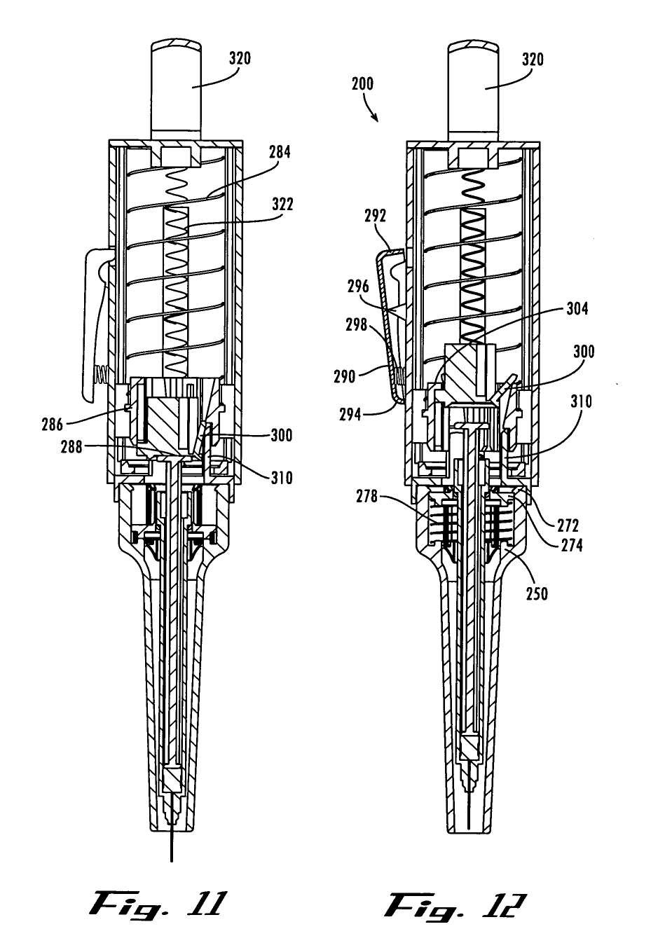

Because the force required to overcome the rearward bias of the return spring 278 and advance the syringe 12 through the barrel 218 is preferably less than the force required to compress the plunger of the syringe 12 and dispense the syringe's contents, the piston 282 preferably extends under the influence of the drive spring 284 to first advance the needle 64 of the syringe outwardly through the opening in the end of the barrel and into the subject's skin (FIG. 10), and continue extending to compress the plunger of the syringe and inject the syringe's contents. Upon reaching its fully extended position wherein the contents of the syringe 12 have been fully dispensed, as shown in FIG. 11, the retraction plunger 288 is released to move rearwardly within the drive sled 286, under the influence of return spring 278, which allows the needle to be withdrawn back into the barrel 218 out of contact with the subject's skin and into a retracted position to prevent inadvertent contact with the contaminated sharp needle. As seen more clearly in FIGS. 11-12, when the piston 282 reaches the end of its forward path of travel, a post 310 projecting from the housing engages the cantilevered arm 300 of the plunger 288 and disengages the arm 300 from the seat 302 of the sled 286. The plunger 288 is then free to move rearwardly under the influence of the return spring 278 and the arm 300, which decompresses along a ramp located within the drive sled, and retracts into contact with the shelf 304 of the drive sled. The post 310 is optionally interchangeable with posts of differing height, or is adjustable in height, to adapt the apparatus to use with different types of syringe, to ensure that the plunger 288 is released at or near the precise point of travel of the drive sled 286 where the syringe plunger is compressed to fully empty the syringe.

The handle portion 216 preferably also has a piston retraction yoke 320 affixed thereto for resetting the drive mechanism to its charged or energized position. One or more springs 322, 324 preferably connect the retraction yoke 320 to the handle 216. At the distal end of the yoke is a loop 326 for the user to grasp and pull to energize the drive mechanism. The bias of the springs 322, 324 is preferably less than that of the drive spring 284. Thus, pulling the yoke 320 rearwardly retracts the piston 282, pulls the drive spring 284 back into compression, and engages the trigger 290. As the piston 282 is pulled back into its retracted position, it contacts the hub 232, which pushes the retraction plunger 288 forward in the drive sled 286 until the resilient cantilevered arm 300 of the retraction plunger reseats against the seat 302 of the drive sled. At this point, the piston portion 282 is energized, and the yoke springs 322 bias the yoke back to its initial position. In an alternate embodiment, the actuation trigger is located on the outer surface of the housing at the proximal end of device beneath the retraction loop 326, so as to prevent inadvertent contact with the actuation button.

In operation, the user opens the receiver portion 260 of the housing 214 and loads a syringe 12 prefilled with a pharmaceutical fluid into the device 12. If the drive mechanism is not energized, the user pulls the loop 326 of the retraction yoke 320 rearwardly to compress the piston portion 282 and drive spring 284, and engage the trigger 290. The rearward force of pulling the piston portion 282 into abutment with the hub 232 advances the retraction plunger 288 within the drive sled 286, and reseats the cantilevered arm 300 in the seat 302. The receiver is closed, and the user places the open end 226 of the device 200 against his or her skin at the desired injection site. The user actuates the trigger to release the drive mechanism, whereupon the piston 282 advances under the influence of drive spring 284. The piston 282 and drive spring 284 overcome the rearward bias of the syringe retraction spring 278, causing the syringe 12 to advance towards the end 226 of the device. The needle 64 of the syringe advances through the opening in the end of the barrel and penetrates the user's skin. The piston 282 continues to advance, driving the plunger 62 into the body 60 of syringe 12 to inject the pharmaceutical therein to the user. As the piston 282 reaches its fully advanced position, the cantilevered protrusions 300 of the retraction plunger 288 are pushed against and parallel to the outer wall of the retraction plunger by posts 310, which releases the retraction plunger to retract within the drive sled. Simultaneously, the advancing bias of the retraction plunger 288 on the syringe 12 is relieved, and the syringe retraction spring 278 retracts the syringe abutment flange 274 back to its resting position, which in turn draws the needle 64 back into the housing 214 of the device 200, as depicted in FIG. 12. The injection is now complete, and the user can open the housing to remove and dispose of the syringe 12. The device can be reloaded with a fresh syringe, and the retraction loop 326 of the piston retraction yoke 320 can be pulled rearwardly to energize drive spring 124 and re-cock the device.

while the invention has been described with reference to preferred and example embodiments, it will be understood by those skilled in the art that a variety of modifications, additions and deletions are within the scope of the invention, as defined by the following claims.

What is claimed is:

1. An apparatus for holding a syringe and injecting fluid from the syringe, said apparatus comprising:
a barrel having a bore extending therethrough for receiving a body portion of the syringe;
a drive spring;
a drive sled movable under the influence of said drive spring to advance the syringe from a retracted position within the barrel to an advanced position wherein a needle portion of the syringe extends outwardly of the barrel, and to compress a plunger portion of the syringe relative to the body portion of the syringe to inject the fluid through the needle portion; and
a retraction plunger movable within the drive sled between an advanced position and a retracted position.

2. The apparatus of claim 1, further comprising a retraction spring for retracting the syringe from the advanced position to the retracted position after injecting the fluid.

3. The apparatus of claim 2, further comprising a syringe retention piston movable between an advanced position and a retracted position under the influence of the retraction spring.

4. The apparatus of claim 3, further comprising an upper receiver wall and a lower receiver wall, wherein the syringe is retained between the syringe retention piston and the upper receiver wall in its retracted position.

5. The apparatus of claim 4, wherein the syringe retention piston compresses the retraction spring against the lower receiver wall when the syringe is in its advanced position.

6. The apparatus of claim 1, wherein the drive sled comprises a pair of arms, each arm having an intermediate barb for retaining the retraction plunger in its advanced position.

7. The apparatus of claim 6, wherein each arm further comprises an inwardly directed free end for retaining the retraction plunger in its retracted position.

8. The apparatus of claim 1, wherein the retraction plunger further comprises a cantilevered arm for engaging the drive sled and holding the retraction plunger in an advanced position in the drive sled.

9. The apparatus of claim 8, wherein the drive sled further comprises a shelf for retaining the retraction plunger in a retracted position.

10. The apparatus of claim 1, further comprising an actuation button for releasing the drive sled from its retracted position.

11. The apparatus of claim 10, wherein the actuation button comprises a plunger setting shaft for resetting the retraction plunger into its advanced position upon compression of the drive spring.

12. The apparatus of claim 1, and further comprising a retraction yoke for retracting the drive sled to an energized position.

13. An apparatus for activating a syringe comprising a housing, a drive mechanism and a syringe retention mechanism, the syringe retention mechanism comprising a syringe retention piston and at least one syringe retraction spring.

14. The apparatus of claim 13, wherein the syringe retention mechanism further comprises an upper receiver wall against which the syringe retention piston is biased by the syringe retraction spring.

15. An apparatus for activating a syringe comprising a housing, a drive mechanism and a syringe retention mechanism, wherein the drive mechanism comprises at least one drive spring for moving a drive sled between an advanced position and a retracted position within the housing, and a retraction plunger movable between an advanced position and a retracted position within the drive sled.

16. The apparatus of claim 15, wherein the drive sled comprises a pair of arms, each arm having an intermediate barb for retaining the retraction plunger in its advanced position.

17. The apparatus of claim 16, wherein each arm further comprises an inwardly directed free end for retaining the retraction plunger in its retracted position.

18. The apparatus of claim 16, wherein the housing comprises at least one plunger retention track for limiting flexure of the arms along a portion of a stroke of travel of the drive sled.

19. The apparatus of claim 15, wherein the housing further comprises at least one resilient retention tab for retaining the drive sled in its retracted position when the drive mechanism is energized.

20. The apparatus of claim 19, further comprising an actuation button for releasing the drive sled from the at least one resilient retention tab.

21. The apparatus of claim 20, wherein the actuation button comprises a plunger setting shaft for resetting the retraction plunger into its advanced position within the drive sled when the drive mechanism is energized.

22. The apparatus of claim 15, wherein the drive sled comprises a seat for engaging an arm of the retraction plunger for retaining the retraction plunger in an advanced position.

23. The apparatus of claim 22, wherein the drive sled further comprises a shelf for retaining the retraction plunger in its retracted position.

24. The apparatus of claim 22, further comprising a post projecting from the housing for disengaging the arm of the retraction plunger from the seat of the drive sled to allow the retraction plunger to retract.

25. The apparatus of claim 24, wherein the post is interchangeable for use with different types of syringe.

26. The apparatus of claim 22, and further comprising a retraction yoke for retracting the drive mechanism to an energized position.
